(19) United States
(12) Patent Application Publication
(10) Pub. No.: US 2026/0041340 A1
(43) Pub. Date: Feb. 12, 2026

(54) IMPLANTABLE DEVICE FOR MONITORING OF HUMAN WELLBEING AND DIFFERENT HEALTH CONDITIONS

(71) Applicant: Imperial College Innovations Limited, London (GB)

(72) Inventors: Salzitsa Yordanova Anastasova-Ivanova, London (GB); Bruno Miguel Gil Rosa, London (GB)

(21) Appl. No.: 18/998,775

(22) PCT Filed: Jun. 30, 2023
(86) PCT No.: PCT/EP2023/068028

§ 371 Date: Jan. 27, 2025

(30) Foreign Application Priority Data
Jul. 28, 2022 (GB) 2211065.4

(51) Int. Cl. A61B 5/1473; A61B 5/00; A61B 5/145; A61B 10/00

(52) U.S. Cl. CPC A61B 5/1473; A61B 5/0028; A61B 5/0031; A61B 5/14532; A61B 5/14539; A61B 5/14546; A61B 10/0012; A61B 2560/0219; A61B 2560/0462

(57) ABSTRACT

Apparatus are disclosed that are arranged to be entirely subcutaneously-implanted, the apparatus comprising an electrochemical sensor for sensing at least one analyte, and a near-field communication (NFC) interface for percutaneous communication of information sensed by the electrochemical sensor.

This disclosure relates to apparatus, devices, systems, and approaches for sensing analytes, and in particular without limitation to an implantable apparatus for sensing analytes within a human body.

### BACKGROUND

Physiological monitoring is fundamental in the diagnostic, prognostic, and evolutionary assessment of many medical conditions (e.g., cardiovascular, neurologic, muscular, endocrine conditions, etc.). Conventionally, parameters and physiological substances related to such conditions are monitored using body surface measurements with equipment such as electrodes.

However, there is a need for improved approaches for on-demand monitoring of analytes within a subject or patient, and in particular for sensing analytes within tissue of a subject.

### SUMMARY

An invention is set out in the claims.

### FIGURES

Specific examples are now described, by way of example only, with reference to the drawings, in which:

FIG. 1 shows a printed circuit board component for an implantable apparatus;

FIG. 2 shows a cross-section of part of an implantable apparatus;

FIG. 3 shows a cross-section of an implantable apparatus;

FIG. 4 shows a scanning electron microscopy image of an implantable apparatus;

FIG. 5 shows a schematic of an electronic circuit for an implantable apparatus;

FIG. 6 shows an exemplary NFC data communication packet;

FIG. 7 shows a method of encrypting data for NFC transmission;

FIG. 8 shows a method of sensing an analyte.

### DETAILED DESCRIPTION

Disclosed herein is an implantable device or apparatus arranged to be entirely subcutaneously-implanted, the apparatus comprising an electrochemical sensor for sensing at least one analyte, and a near-field communication, NFC, interface for percutaneous (or trans-cutaneous) communication of information sensed by the electrochemical sensor.

Conventional commercially-available implants are used for the detection and/or delivery of electric stimulation through bio-potential channels (or electrodes), and typically do not perform electrochemical sensing of analytes. Conventional implants are typically battery-powered.

The implantable devices and/or apparatuses disclosed herein are beneficially arranged to be implanted in subcutaneous tissue for electrochemical sensing of analytes, providing physiological data sensing at the tissue level. In particular, the implantable devices and/or apparatuses may be entirely subcutaneously implanted, such as entirely under the skin of a human body, such as within a torso of a human body. The sensing provided by the implantable apparatus thus provides “supra-cell” monitoring of chemical and biological signals produced at the tissue level, such as within interstitial fluids. The presence or concentration of a chemical and/or biological analyte may be detected and/or measured. In some examples, the analytes targeted are indicative of human fertility.

Furthermore, the approaches used herein make use of an NFC interface. Advantageously, the NFC interface allows information sensed by the electrochemical sensor to be communicated percutaneously, i.e. across the skin barrier. The NFC interface also allows the implantable apparatus to receive power wirelessly from an external source, rather than rely on batteries. The external source may be a mobile phone or other NFC interface that is arranged to power the implantable apparatus and to receive data communicated from the implantable apparatus using NFC. The implantable apparatus may include a microcontroller arranged to encrypt data. The NFC interface enables the receipt of power percutaneously, i.e. across the skin of the subject, when the implantable apparatus is entirely subcutaneously implanted, and the NFC interface is also arranged to wirelessly transmit data percutaneously.

The term ‘chemical sensor’ is used herein to also refer to types of electrochemical sensor.

The implantable apparatus may be based on, and/or comprise a printed circuit board.

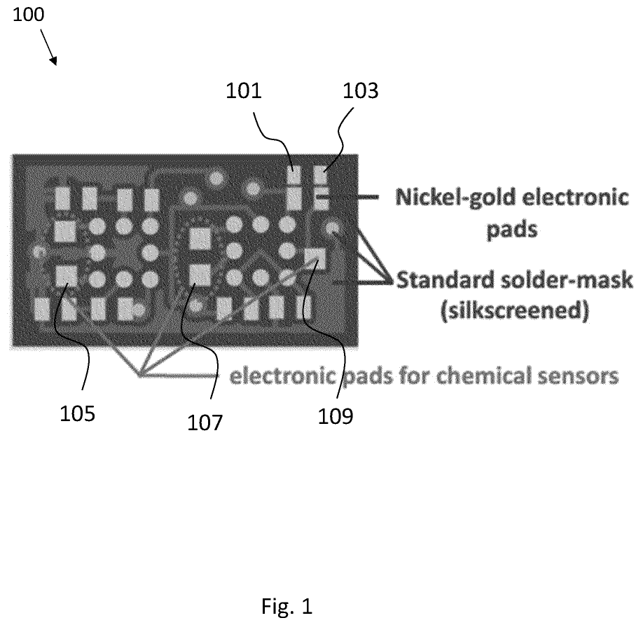

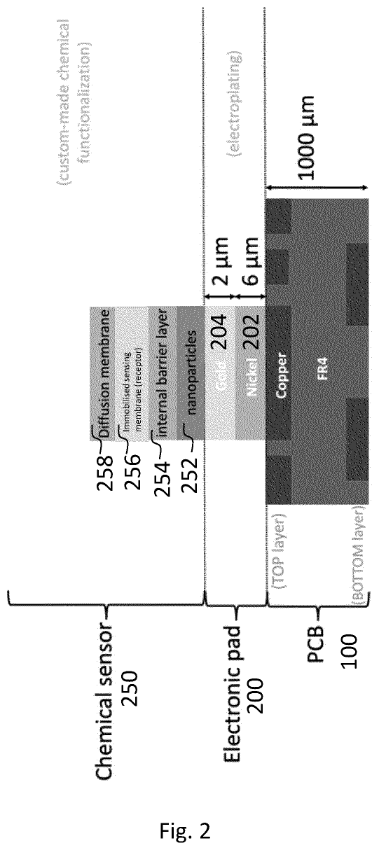

FIG. 1 shows an example printed circuit board 100. The printed circuit board (PCB) is based on conventional materials such as FR4 and copper. Components of the implantable apparatus may be mounted to the printed circuit board 100, such as at electronic pads 101, 103. Further electronic pads 105, 107, 109 are each functionalised to act as chemical, or electrochemical, sensors, as described further below in relation to FIG. 2.

Each electronic pad 101, 103, 105, 107, 109 may have a nickel-gold, or simply gold, layer deposited on it, which is arranged to act as an electrically conductive layer. Each electronic pad 101, 103, 105, 107, 109 of the PCB 100 does not have a solder-mask process applied to it as would be the case for a conventional PCB and as may the case for other portions of the PCB. Instead, the gold and/or nickel are simply applied to exposed copper of the PCB 100.

Components of the implantable apparatus that may be mounted to the printed circuit board 100 include an NFC tag, an NFC antenna, a microcontroller, at least one amplifier, and at least one resistor. Each component will be further explained in relation to the further examples below. Any component may be mounted on either side of the PCB. In some examples, only one side of the PCB may be used in this way, and in other examples, both sides of the PCB may be used in this way.

The PCB 100 also features conductive traces 111 that are covered by a standard solder-mask (silkscreened) process. As will be appreciated, the conductive traces 111 not shown in FIG. 1 allow different components to be connected across the PCB. The copper traces may have a width of 0.1 mm. The PCB may likewise comprise copper pools and copper-plated vias.

FIG. 2 shows a cross-section of part of the implantable apparatus. The printed circuit board 100 of FIG. 1 is shown in FIG. 2 and has an exemplary thickness of 1000 μm. An electronic pad 200 comprising a nickel layer 202 with exemplary thickness of 6 μm and a gold layer 204 with an exemplary thickness of 2 μm is arranged on top of the PCB. The electronic pad may be formed on top of an exposed area of copper of the PCB by a technique such as electroplating. The nickel layer is arranged on top of the copper of the PCB and the gold layer is arranged on top of the nickel layer. In other examples, the nickel layer may be omitted, or different thicknesses or ordering of layers may be used.

A chemical sensor 250 is formed on the electronic pad. The chemical sensor comprises four layers: nanoparticles 252, an internal barrier layer 254, an immobilised sensing membrane, or receptor, 256, and a diffusion membrane 258. The electronic pad is thus ‘functionalised’ as a chemical sensor for sensing an analyte, as in the electronic pads 105, 107, 109 of FIG. 1. Not shown in FIG. 2 are pads for connecting electrical components such as an amplifier to the PCB. Those pads may also have nickel and gold layers arranged on top of them like the example of FIG. 2. The mixture and thickness of the gold and nickel layers dictates how good the electrical connection will be with the functionalized sensing layers placed on top.

The diffusion membrane 258 of the chemical sensor 250 allows the analyte to diffuse into the sensor. The analyte diffuses across the diffusion membrane 258 layer and is held at the immobilised sensing membrane 256. Here the analyte interacts with the chemical sensor 250 so that an electrical change or signal is produced. The electrical signal is enhanced by the presence of the nanoparticles 252.

In each example, the functionalised chemical sensor may function as an electrode in a sensing and/or measurement circuit, where the “electrode” has been functionalized to respond to chemical analytes through the deposition of specific sensor layers (as described previously) by an electrochemical process. The chemical sensor may be formed in order to target, or sense, particular chemical and/or biological analytes, such as pH, sodium, calcium, potassium, lactate, glucose, cortisol, estrogen, progesterone, and/or luteinizing hormone (LH).

The materials and thicknesses of the layers of the functionalised chemical sensor are chosen to achieve the best sensitivity for the chemical analyte being targeted. Example materials are shown in Table 1. For example, to detect pH, the diffusion membrane material may be chitosan, the immobilised sensing membrane (receptor material) may be iridium oxide nanoparticles, and so on.

TABLE 1

| | | Immobilised | | |
|---|---|---|---|---|
| | | sensing | Internal | |
| Examples of | Diffusion | membrane | barrier | |
| materials for | membrane | (receptor) | layer | Nanoparticles |
| detection of . . . | material | material | material | material |
| pH | chitosan | Iridium | copolymer of | Poly(3,4- |
| | | oxide | sulphonated | ethylenedioxythiophene) - |
| | | nanoparticles- | polyesther | poly(styrenesulfonate) |
| | | | ether | |
| | | | sulphone - | |
| | | | polyether | |
| | | | sulphone | |
| | | | (SPEES/PES) | |
| | | | membrane | |
| Sodium | chitosan | Selectophore | copolymer of | Poly(3,4- |
| | | grade | sulphonated | ethylenedioxythiophene) - |
| | | sodium | polyesther | poly(styrenesulfonate) |
| | | ionophore X | ether | |
| | | | sulphone - | |
| | | | polyether | |
| | | | sulphone | |
| | | | (SPEES/PES) | |
| | | | membrane | |
| Calcium | chitosan | Calcium | copolymer of | Poly(3,4- |
| | | ionophore II | sulphonated | ethylenedioxythiophene) - |
| | | | polyesther | poly(styrenesulfonate) |
| | | | ether | |
| | | | sulphone - | |
| | | | polyether | |
| | | | sulphone | |
| | | | (SPEES/PES) | |
| | | | membrane | |
| Potassium | chitosan | valinomycin | copolymer of | Poly(3,4- |
| | | (potassium | sulphonated | ethylenedioxythiophene) - |
| | | ionophore) - | polyesther | poly(styrenesulfonate) |
| | | | ether | |
| | | | sulphone - | |
| | | | polyether | |
| | | | sulphone | |
| | | | (SPEES/PES) | |
| | | | membrane | |
| Lactate | 2%, 5%, 7%, | Lactate | copolymer of | Gold |
| | Polyurethane | oxydase | sulphonated | nanoparticles |
| | 1% nylon | and 1- | polyesther | anchored on |
| | | lactate | ether | reduced |
| | | dehydrogenase | sulphone - | graphene |
| | | (LDH) | polyether | oxide |
| | | | sulphone | |
| | | | (SPEES/PES) | |
| | | | membrane | |
| Glucose | 2%, 5%, 7%, | Glucose | copolymer of | Gold |
| | Polyurethane | Oxidase | sulphonated | nanoparticles |
| | 1% nylon | | polyesther | |
| | | | ether | |
| | | | sulphone - | |
| | | | polyether | |
| | | | sulphone | |
| | | | (SPEES/PES) | |
| | | | membrane | |
| Cortisol | 2%, 5%, 7%, | Cortisol | copolymer of | Gold |
| | Polyurethane | antibody | sulphonated | nanoparticles/ |
| | 1% nylon | | polyesther | molybdenum |
| | | | ether | disulphide/gold |
| | | | sulphone - | nanoparticles |
| | | | polyether | |
| | | | sulphone | |
| | | | (SPEES/PES) | |
| | | | membrane | |
| LH | 2%, 5%, 7%, | luteinizing | copolymer of | PEG-coated |
| | Polyurethane | hormone- | sulphonated | magnetite |
| | 1% nylon | releasing | polyesther | nanoparticles |
| | | hormone | ether | |
| | | | sulphone - | |
| | | | polyether | |
| | | | sulphone | |
| | | | (SPEES/PES) | |
| | | | membrane | |
| Estrogen | Polyethersulfone | Estrogen | copolymer of | Gold |
| | microfiltration | receptor | sulphonated | nanoparticles |
| | membrane | alpha and | polyesther | |
| | | beta | ether | |
| | | | sulphone - | |
| | | | polyether | |
| | | | sulphone | |
| | | | (SPEES/PES) | |
| | | | membrane | |
| Progesterone | Poly(3- | Progesterone | copolymer of | Colloidal |
| | hydroxybutyrate) | receptor | sulphonated | gold |
| | biodegradable | membrane | polyesther | nanoparticles |
| | polymeric | component-1 | ether | |
| | membrane | | sulphone - | |
| | | | polyether | |
| | | | sulphone | |
| | | | (SPEES/PES) | |
| | | | membrane | |

Using the nanoparticles 252 as a component of the sensing layer significantly improves the sensitivity of the electrochemical sensor. Nanoparticles are employed for electrode surface modification, signal molecular labelling (specific target binding) and signal amplification, as well as being used also as catalysts for the chemical reactions in progress. Nanoparticles also provide good biocompatibility and higher surface activity area over the electrodes, therefore improving the ability of electrodes to transfer electrons, the immobilization of bioactive substances on the electrode surface and shortening of the detection time. The nanoparticles 252 to be used can have different sizes (within the nanometre range), shapes (spherical, cylindrical, planar, etc.) and can be made of noble metal nanomaterials (e.g., gold and silver), semiconductor materials (quantum dots), carbon nanomaterials (carbon nanotubes), graphene oxide and composite nanomaterials. As such, there are multiple methods for deposition of nanoparticles depending on the type of nanomaterial employed and substrate surface to which they attach. Examples include dip coating, spin coating, solvent evaporation, chemical vapour deposition and transfer printing.

In some examples, the nanoparticles 252 may be omitted, and the chemical sensor 250 may comprise three layers: an internal selective layer, a middle sensing layer, and an external biocompatible layer.

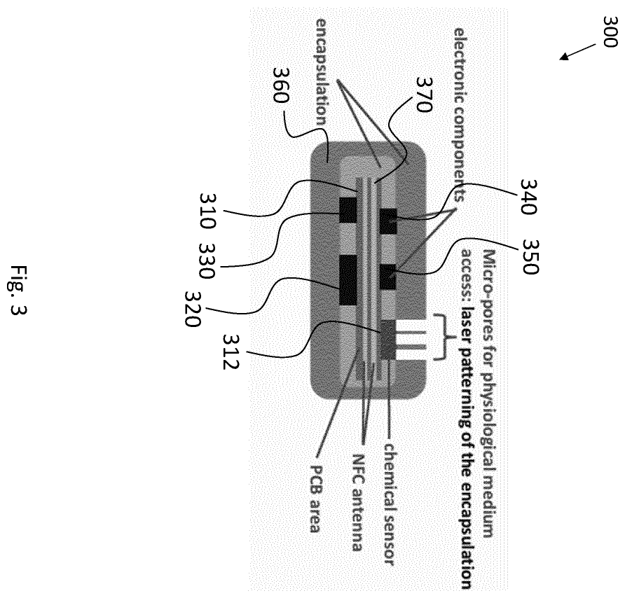

FIG. 3 shows a cross-section of an implantable apparatus 300. The implantable apparatus 300 is based on a PCB 310 such as the PCB 100 of FIGS. 1 and 2. As in FIGS. 1 and 2, a chemical sensor 312 is formed on an electronic pad the PCB. Electronic components 320, 330, 340, 350, such as an amplifier, resistor, and/or NFC tag, are also mounted to electronic pads of the PCB.

The implantable apparatus 300 features an outer layer of encapsulation 360. The encapsulation 360 is made of a biocompatible material and ensures that the implantable apparatus 300 is not rejected by the body of the subject and remains functional. The encapsulation 360 is provided with micro-pores to allow the chemical sensor(s) access to the physiological medium. The micro-pores may be created by laser patterning of the encapsulation 360 or an alternative suitable technique.

The implantable apparatus 300 also comprises an NFC antenna 370. In an example, the NFC antenna 370 is wound around the PCB 310.

NFC technology allows energy transfer between the implantable apparatus and an NFC-enabled external device that comes within sufficiently close range. Hence, no wiring, no batteries and no separate electronics unit is needed to power the implantable apparatus. The NFC antenna 370 instead harvests power or energy from an incident field.

Any device equipped with an NFC chip can power the implantable apparatus. Preferably, the use of encryption prevents unauthorised devices from accessing data from the device.

Although only one chemical sensor 312 is shown in the example of FIG. 3, in other examples, the implantable apparatus may feature multiple chemical sensors. Each of the multiple chemical sensors may be of the same type, or of a different type. For example, one chemical sensor may be arranged to perform amperometry, and another chemical sensor may be arranged to perform voltammetry. Hence, the implantable apparatus is capable of performing the two techniques of amperometry and voltammetry and is further capable of performing the two techniques simultaneously. The implantable apparatus may also be arranged to perform impedance-based detection of analytes.

With such apparatuses, two or more different analytes may be measured at once. Examples of analytes that can be measured by the apparatuses and devices disclosed herein are as follows: pH, sodium, calcium potassium, lactate, luteinizing hormone, glucose, cortisol, estrogen, and progesterone. In examples in which the implantable apparatus is arranged to perform amperometry and voltammetry, the analytes may be arranged to simultaneously sense at least one of pH, sodium, calcium, potassium (using voltammetry); and one of lactate, glucose, cortisol.

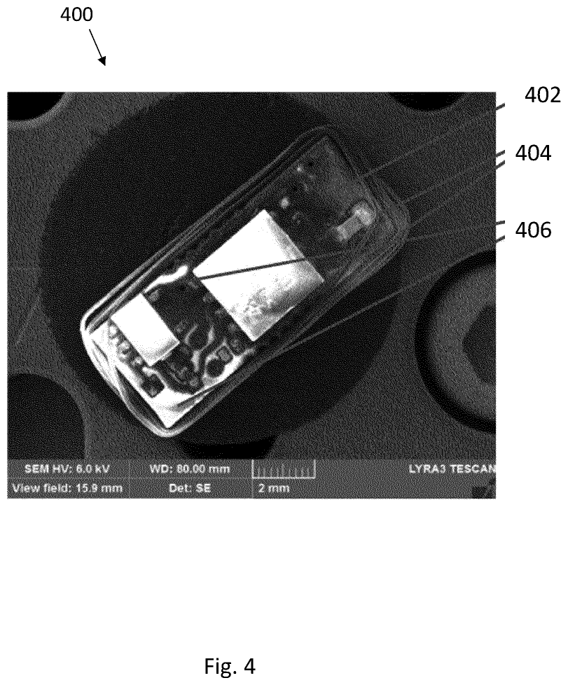

FIG. 4 shows an example implantation of the implantable apparatuses disclosed herein. FIG. 4 is a scanning electron microscope (SEM) image 400 of such an implantable apparatus. A PCB 402 is shown, featuring mounted electronic components. Also shown is an NFC antenna 404, which is formed of copper wire wrapped around the external perimeter of the PCB in 20 loops. Also shown in contrast is an encapsulation layer 406. In this example, the encapsulation layer comprises polymethyl siloxane (PDMS, 1 mm thick) and parylene (100 μm thick). Both of those materials are biocompatible. In other examples, other biocompatible materials may be used for the encapsulation layer. Advantageously, the encapsulation layer 406 protects the interior components of the implantable apparatus and increases the lifetime of the apparatus.

As an example, the implantable apparatus may be based on a PCB with a surface area of 3.7 mm by 6.5 mm. In some examples, the implantable apparatus may be injected into the target tissue through a needle, thus alleviating some technical constraints posed by medical surgery approaches.

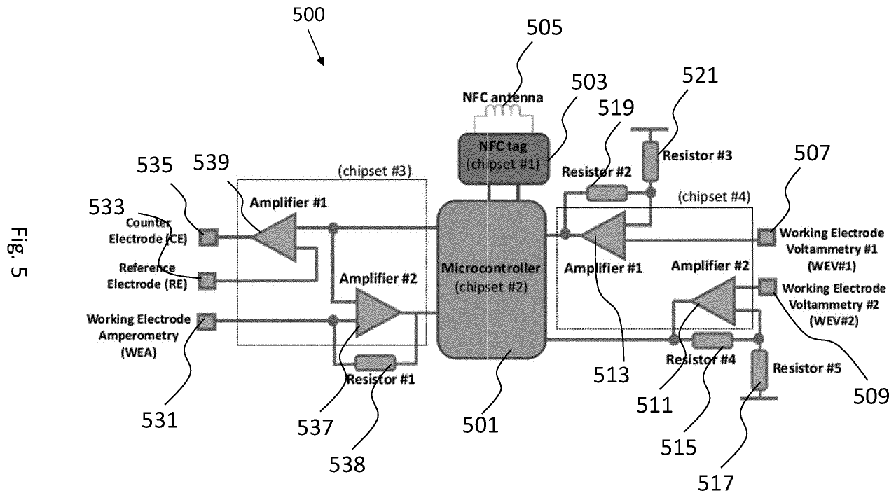

FIG. 5 shows a schematic of an electronic circuit 500 for an implantable apparatus. The electronic circuit 500 is suitable for implementation on or with the PCB-based implantable apparatuses disclosed herein.

The electronic circuit 500 comprises a microcontroller 501. The microcontroller is arranged to control the other components of the electronic circuit 500 and to process information for communication thereby. For example, the microcontroller is arranged to interact with an NFC tag 503. The NFC tag 503 is arranged to transmit data using the NFC communication protocol, and the microcontroller may be used to encrypt that data. Together with an NFC antenna 505, the NFC tag 503 forms an NFC interface. The NFC antenna 505 allows the circuit 500 to wirelessly receive power from an external source. In other words, there are no active power sources (such as batteries) within the electronic circuit 500, which may be considered a passive circuit in that regard.

The electronic circuit 500 is arranged to perform voltammetry using a first voltammetry working electrode 507 and a second voltammetry working electrode 509. The working voltammetry electrodes may be functionalised into chemical sensor(s) as described herein, such as in the example of FIG. 2. The voltammetry portion of the electronic circuit 500 also comprises a first amplifier 511 and a second amplifier 513 for each voltammetry working electrode 507, 509. The amplifiers may be mounted on a PCB as described herein. Each amplifier 511, 513 is connected to resistor components 515, 517, 519, 521 of appropriate values, as will be appreciated by the skilled person, and is arranged to amplify electrical signal produced by the working electrodes 507, 509. A commercial off-the-shelf dual-amplifier may be used for the amplifiers 511, 513.

The electronic circuit 500 is also arranged to perform amperometry, and in some examples may perform amperometry simultaneously with voltammetry. The electronic circuit 500 is arranged to perform amperometry using a first amperometry working electrode 531, a reference electrode 533, and a counter electrode 535. In some examples, the three electrodes 531, 533, 535 may be considered to form a single chemical sensor, and may be functionalised as described herein, such as in the examples of FIGS. 2 and 3. The first amperometry working electrode is connected to an amplifier 537, which also has a resistor 538 connected across it. The reference electrode 533 and the counter electrode 535 are connected to another amplifier 539. A commercial off-the-shelf dual-amplifier may be used for the amplifiers 537, 539. The amplifiers 537, 539 are arranged to amplify electrical signals produced by the electrode(s).

Referring back to the chemical sensor 312 of FIG. 3, such a chemical sensor may in some examples be arranged to be divided into different “electrodes” corresponding to those of the electronic circuit 500. For example, the chemical sensor may be arranged to provide three electrodes for amperometry as described above in relation to the electronic circuit 500: a first amperometry working electrode 531, a reference electrode 533, and a counter electrode 535. An analogous arrangement may be used for voltammetry with the same chemical sensor or an additional chemical sensor. In such examples, the encapsulation 360 is structured correspondingly with micro-pores so that the distinct “electrodes” of the chemical sensor 312 can access the interstitial fluid and/or tissue that is being sensed.

In some examples, the reference electrode 533 and the counter electrode 535 may be used in combination with the voltammetry electrodes in order to produce a signal based on the presence of an analyte.

The electronic circuit 500 shown in FIG. 5 is capable of measuring up to three chemical analytes at the same time. One acquisition channel is mounted using the amperometry circuit topology described above (targeting e.g. lactate or glucose, cortisol, etc.) and a further two acquisition channels are mounted on a voltammetry circuit topology as described above (targeting e.g. pH or sodium, potassium, calcium, etc.). Each acquisition channel is connected to an individual working electrode, with the counter and reference electrodes shared by the three acquisition channels. Each acquisition channel may be described as an ionic sensitive channel.

The electronic circuit 500 is arranged to amplify and convert the voltage and/or current signals from the channels into digital samples to be acquired by the embedded microcontroller before secure wireless transmission using the NFC protocol.

As will be appreciated, components of the electronic circuit 500 may be varied. What is important is that the electronic circuit 500 uses chemical sensors arranged on the electrodes in order to perform voltammetry and/or amperometry of analytes and then to communicate measured data using NFC. The electronic circuit 500 may alternatively or additionally be arranged to perform an impedance detection of at least one analyte.

In some examples, the electronic circuit 500 can sense any pair combination of voltammetry (pH, sodium, calcium, potassium, etc.) and amperometry (lactate, glucose, cortisol, etc.) analytes, provided that the electrodes are chemically functionalized to respond to such stimulus.

In some examples, miniaturisation of the sensor means that the different sensing parts can sense the same or different analytes.

The electronic circuit 500 is also arranged to implement cross-checking between sensors. Metrics for signal magnitude, interference and drift are defined within programming code running inside the microcontroller. Deviations from the typical calibration curves for each single analyte and the influence of external factors (such as temperature) are also recorded and may be transmitted wirelessly to the user.

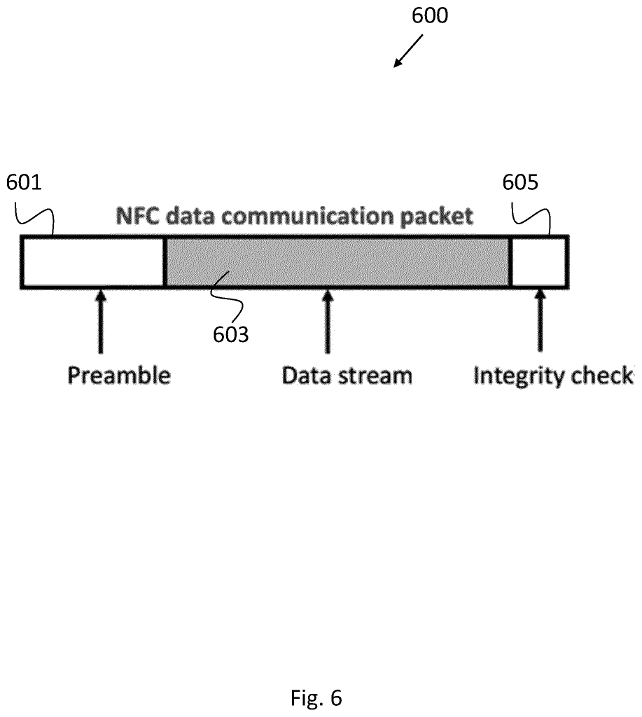

FIG. 6 shows an example of an NFC data communication packet 600 suitable for transmitting data over the NFC interface of the implantable apparatuses disclosed herein. Data may be transmitted or received using the NFC tag of the NFC interface and power may be received using the NFC antenna of the NFC interface.

The NFC interface is arranged to interface with devices such as a tablet or mobile phone for power harvesting and data communication.

The NFC data communication packet 600 comprises a preamble 601, a data stream 602, and an integrity check field 605.

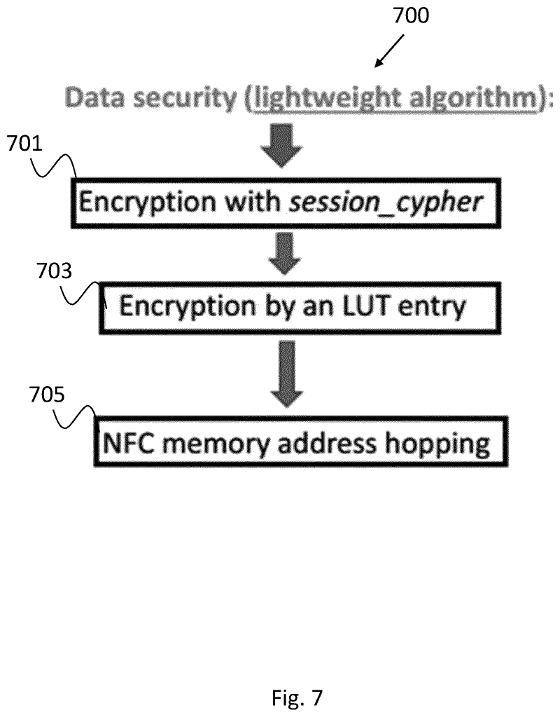

The preamble 601 comprises a universal data preamble that functions as a hand-shaking protocol between the two entities involved in the communication. The data stream 602 comprises the payload, or data proper, that is being sent. The data stream 602 field may be 1 kB in size in a plain format, or encrypted. As will be discussed below in relation to the example of FIG. 7, the payload may be encrypted inside the microcontroller and, therefore, cannot be decoded by other NFC receivers unless they know the decoding cypher. Only the data preamble 601 is completely transparent between NFC entities.

The integrity check field 605 is a 16-bit cyclic redundancy check to avoid transmission and reception of corrupted data packets.

The fact that the NFC protocol generates a radiofrequency signal to send the communication packets means that the energy released by this process can be used by other NFC close-by devices for wireless recharging (energy harvesting). The implantable apparatus itself may be powered this way. The NFC antenna of the implantable apparatuses disclosed herein is arranged such that the implantable apparatus can harvest sufficient wireless energy from the RF field produced by a mobile phone at gap distances up to 2 cm for example.

In some examples, the NFC communication capability of the NFC interface is based on the standard communication protocol established for NFC (ISO/IEC 14443 and ISO/IEC 18000-3), so any external device complying with this standard can power the implantable device.

The NFC communication protocol allows the implantable apparatus to transfer larger amounts of data than, for example, RFID communication, inside a single transmission packet. Using NFC, 1 kB worth of physiological data may be exchanged in each exchange with the external device, whereas current RFID technology is merely capable of transferring a few dozens of bytes, half of each being employed to transmit the chip identification number over air and signal communication flags. Moreover, the radiofrequency power strength allocated to RFID technology is lower than NFC, which translates to smaller communication distances between the implantable apparatus and the external device.

FIG. 7 shows an example method 700 for encrypting data, such as the measurement data provided by the chemical sensors. The encrypted data is then provided for transmission using the NFC interface. The method 700 for encrypting data is a lightweight algorithm, i.e. it is intended for low-power implementation on the implantable apparatuses disclosed herein. The method 700 provides that data transmitted by the NFC tag of the implantable apparatus can only be decoded by an authorised external device. An authorised external device may, in some examples, be provided with a synchronised application.

The method 700 is divided into three sequential protection layers and generates approximately 2 to the power of 45 code combinations.

At a first step 701, the method comprises encrypting the data with a session_cypher step: an XOR operation between an 8-bit fixed cypher variable shared by the implantable apparatus and the authorised external device (in some examples, through a synchronised application on a mobile phone) and a one-time random cypher variable generated by the implantable apparatus in every NFC transmission. A “synchronised” same fixed cypher with the implantable application shares the apparatus, thereby enabling it to decipher the encrypted data sent by the implantable apparatus. In some implementations, only a single implantable apparatus and a single application share a unique fixed cypher. The fixed cypher is hard coded once to the non-volatile memory of the implantable apparatus (inside the microcontroller) during device programming at the fabrication stage and, also, within the programming code of the application before installation on the selected external device, thus becoming “synchronised” with the implantable apparatus. A specification form and software template may be used by a certified technician to install the “synchronised” application inside a single external device, with both the form and software template being subsequently destroyed to prevent their usage on other external devices. Similar applications that do not share the same fixed cypher as a specific implantable apparatus are thus “unsynchronised” and unable to decode data from the implantable apparatus.

At a second step 702, the method comprises encrypting the data by a look-up table (LUT) entry. The second step 702 comprises an XOR operation between a single 16-bit pattern contained inside an LUT which contains 64 different pattern entries. The LUT is hard-coded to a non-volatile memory of the implantable apparatus and synchronised application and it is unique to each implantable apparatus. The particular LUT entry of the 64 different entries used during encryption by the implantable apparatus is recovered by the “synchronized” application by manipulation of the bit stream coded by a combination of parts of the fixed cypher and session cypher. The number of entries, 64, is used as an example based on an exemplary non-volatile memory capacity of the implantable apparatus microcontroller. In this example, the 64 entries occupy (2\hat{ }6*×16-bit/8)=128 bytes or 128 address positions. In other examples, fewer or greater number of entries may be used according to the available memory capacity.

At a third step 705, the method comprises NFC memory address hopping. The third step 705 comprises the random allocation of chunks of data bits into non-continuous sequential address blocks inside the NFC tag before transmission. A single communication channel is used and the entire data stream is divided into chunks of bits that are allocated to different random address positions inside the NFC tag memory, thus breaking the continuity of the data stream in every transmission. The original continuous stream can only be put together by the synchronised external device.

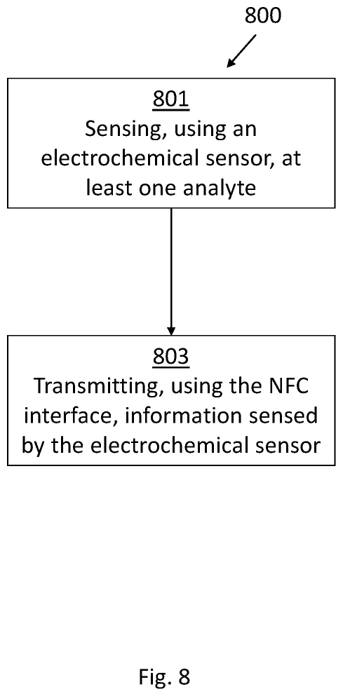

FIG. 8 shows a method 800 of sensing an analyte, the method to be performed using the implantable apparatuses and devices disclosed herein, such as those of the examples of FIGS. 2 to 7, when entirely subcutaneously implanted.

At a first step 801, the method comprises sensing, using the electrochemical sensor, at least one analyte.

At a second step 803, the method comprises transmitting, using the NFC interface, information sensed by the electrochemical sensor.

As described above in relation to the arrangement of the implantable apparatus, the method may further comprise simultaneously sensing, using the electrochemical sensor, more than one analyte.

As described above in relation to the arrangement of the implantable apparatus, the method may further comprise simultaneously performing voltammetry and amperometry.

As described above in relation to the arrangement of the implantable apparatus, the method may further comprise sensing at least one of pH, sodium, calcium potassium, lactate, luteinizing hormone, glucose, cortisol, estrogen, and progesterone.

As described above in relation to the arrangement of the implantable apparatus, the method may further comprise simultaneously sensing one of: pH, sodium, calcium, potassium, and one of: lactate, glucose, cortisol.

As described above in relation to the arrangement of the implantable apparatus, the method may further comprise harvesting, using the NFC interface, power from an electromagnetic field.

As described above in relation to the arrangement of the implantable apparatus, the method may further comprise sensing at least one analyte indicative of human fertility.

As described above in relation to the arrangement of the implantable apparatus, the method may further comprise encrypting the information sensed by the electrochemical sensor prior to the percutaneous communication. In some examples, the encryption is performed using the method 700 of FIG. 7.

1. An apparatus arranged to be entirely subcutaneously-implanted, the apparatus comprising:
an electrochemical sensor for sensing at least one analyte, and
a near-field communication, NFC, interface for percutaneous communication of information sensed by the electrochemical sensor.

2. The apparatus of claim 1, wherein the electrochemical sensor is arranged to simultaneously sense more than one analyte.

3. The apparatus of any preceding claim, wherein the NFC interface is a passive component.

4. The apparatus of any preceding claim, wherein the electrochemical sensor comprises a selective layer, a sensing layer, and an external biocompatible layer.

5. The apparatus of any preceding claim, wherein the electrochemical sensor is arranged on a pad of a printed circuit board.

6. The apparatus of claim 5, wherein the pad of the printed circuit board is not covered by any solder mask.

7. The apparatus of claim 5 or claim 6, wherein the pad comprises a contact layer comprising gold.

8. The apparatus of claim 7, wherein the contact layer further comprises nickel.

9. The apparatus any preceding claim, wherein the electrochemical sensor is arranged to simultaneously perform voltammetry and amperometry.

10. The apparatus of any preceding claim, wherein the apparatus is arranged to perform impedance detection of the at least one analyte.

11. The apparatus of any preceding claim, wherein the electrochemical sensor is arranged to sense at least one of pH, sodium, calcium potassium, lactate, luteinizing hormone, glucose, cortisol, estrogen, and progesterone.

12. The apparatus of any preceding claim, wherein the electrochemical sensor is arranged to simultaneously sense:
one of: pH, sodium, calcium, potassium; and
one of: lactate, glucose, cortisol.

13. The apparatus of any preceding claim, wherein the NFC interface is arranged to harvest power from an electromagnetic field.

14. The apparatus of any preceding claim, wherein the apparatus does not comprise a battery.

15. The apparatus of any preceding claim, wherein the electrochemical sensor is arranged to sense at least one analyte indicative of human fertility.

16. The apparatus of any preceding claim, further comprising an amplifier arranged to amplify a signal produced by the electrochemical sensor.

17. The apparatus of any preceding claim, the apparatus being arranged to encrypt the information sensed by the electrochemical sensor prior to the percutaneous communication.

18. A method of sensing an analyte, the method performed in the apparatus of any preceding claim when the apparatus is entirely subcutaneously-implanted, the method comprising:
sensing, using the electrochemical sensor, at least one analyte; and
transmitting, using the NFC interface, information sensed by the electrochemical sensor.

19. The method of claim 18, further comprising simultaneously sensing, using the electrochemical sensor, more than one analyte.

20. The method of claim 18 or claim 19, further comprising simultaneously performing voltammetry and amperometry.

21. The method of any of claims 18 to 20, further comprising sensing at least one of pH, sodium, calcium potassium, lactate, luteinizing hormone, glucose, cortisol, estrogen, and progesterone.

22. The method of any of claims 18 to 21, further comprising simultaneously sensing:
one of: pH, sodium, calcium, potassium; and
one of: lactate, glucose, cortisol.

23. The method of any of claims 18 to 22, further comprising harvesting, using the NFC interface, power from an electromagnetic field.

24. The method of any of claims 18 to 23, further comprising sensing at least one analyte indicative of human fertility.

25. The method of any of claims 18 to 24, further comprising encrypting the information sensed by the electrochemical sensor prior to the percutaneous communication.
